(19) United States
(12) Patent Application Publication
(10) Pub. No.: US 2009/0275611 A1
(43) Pub. Date: Nov. 5, 2009

(54) Compounds Which Modulate The CB2 Receptor

(75) Inventors: Doris Riether, Newtown, CT (US); David Smith Thomson, Ridgefield, CT (US); Lifen Wu, New Milford, CT (US); Renee M. Zindell, New Milford, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/293,484

(22) PCT Filed: Apr. 2, 2007
(86) PCT No.: PCT/US07/65749

§ 371 Date: Sep. 18, 2008

Related U.S. Application Data
(60) Provisional application No. 60/744,446, filed on Apr. 7, 2006.

(51) Int. Cl. A61K 31/47; C07C 211/55; A61K 31/136; A61P 29/00; A61P 9/00; A61P 17/00; A61P 37/08; A61P 1/16; A61P 1/00; A61P 27/02; A61P 25/00; A61P 9/10; A61P 9/04; C07D 213/74; A61K 31/44; C07C 381/00; C07D 215/12; C07C 217/00; A61K 31/135; C07D 213/643; A61K 31/4412

(52) U.S. Cl. 514/311; 564/434; 514/653; 546/312; 514/352; 564/341; 546/176; 564/389; 514/649; 546/300; 514/351

(57) ABSTRACT

Compounds are provided which bind to and are agonist, antagonists or inverse agonists of the CB2 receptor, the compounds having the general formula (I) wherein R₁, R₂, R₃, X and Ar have the meanings given in the specification, and the preparation and use thereof. The compounds are valuable CB2 receptor modulators, and are useful for treating inflammation. Those compounds which are agonists are additionally useful for treating pain.

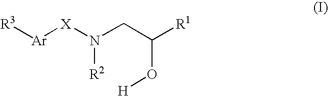

### APPLICATION DATA

This application claims benefit to U.S. provisional application 60/744,446 filed Apr. 7, 2006.

### BACKGROUND OF THE INVENTION

1. Technical Field

The present invention relates to novel compounds which modulate the CB2 receptor and their use as medicaments.

2. Background Information

Cannabinoids are a group of about 60 distinct compounds found in Cannabis sativa (also know as marijuana) with cannabinol, cannabidiol and Δ⁹-tetrahydrocannabinol (THC) being the most representative molecules. The therapeutic usage of Cannabis can be dated back to ancient dynasties of China and includes applications for various illnesses ranging from lack of appetite, emesis, cramps, menstrual pain, spasticity to rheumatism. The long history of Cannabis use has led to the development of several pharmaceutical drugs. For example, Marinol and Cesamet which are based on THC and its analogous nabilone, respectively, are used as anti-emetic and appetite stimulant. Despite of the clinical benefits, the therapeutic usage of cannabis is limited by its psychoactive effects including hallucination, addiction and dependence. Mechoulam R, ed. Cannabinoids as Therapeutic Agents, Boca Raton, Fla.; CRC Press, 1986 provides a review of the medicinal use of cannabis.

The physiological effects of cannabinoids are mediated by at least two G-protein coupled receptors, CB1 and CB2. Autoradiographic studies have demonstrated that CB1 receptors are expressed primarily in the central nervous system, specifically in the cerebral cortex, hippocampus, basal ganglia and cerebellum. They are also found in the reproductive system and other peripheral tissues including that of the immune system, but to a lesser degree. CB1 receptors regulate the release of neurotransmitters from the pre-synaptic neurons and are believed to mediate most of the euphoric and other central nervous system effects of cannabis, such as THC-induced ring-catalepsy, hypomobility, and hypothermia, which were found to be completely absent in mice with a deletion of the CB1 gene (Zimmer et al., Increased mortality, hypoactivity, and hypoalgesia in cannabinoid CB1 receptor knockout mice. Proc Natl Acad Sci USA. (1999) 96:5780-5785.)

CB2 receptors are almost exclusively found in the immune system, with the greatest density in the spleen. It is estimated that the expression level of CB2 in the immune cells is about 10 to 100 times higher than CB1. Within the immune system, CB2 is found in various cell types, including B cells, NK cells, monocytes, microglial cells, neutrophils, T cells, dentritic cells and mast cells, suggesting that a wide range of immune functions can be regulated through CB2 modulators (Klein et al., The cannabinoid system and immune system. J Leukoc Biol (2003) 74:486-496). This is supported by the finding that the immunomodulatory effect of THC is absent in CB2 deficient mice mice (Bicklet et al., Immunomodulation by cannabinoid is absent in mice deficient for the cannabinoid CB2 receptor. Eur J Pharmacol (2000) 396:141-149). CB2 selective ligands have been developed and tested for their effects in various imflammatory settings. For example, in animal models of inflammation, CB2 selective agonists, inverse agonists and antagonists have been shown to be effective in suppressing inflammation (Hanus et al., HU-308: a specific agonist for CB(2), a peripheral cannabinoid receptor. Proc Natl Acad Sci USA. (1999) 96:14228-14233, Ueda et al., Involvement of cannabinoid CB(2) receptor-mediated response and efficacy of cannabinoid CB(2) receptor inverse agonist, JTE-907, in cutaneous inflammation in mice. Eur J. Pharmacol. (2005) 520:164-171 and Smith et al., The anti-inflammatory activities of cannabinoid receptor ligands in mouse peritonitis models Eur J Pharmacol. (2001) 432:107-119.). Furthermore, CB2 selective agonists inhibit disease severity and spasticity in animal models for multiple sclerosis (Baker et al., Cannabinoids control spasticity and tremor in a multiple sclerosis model. Nature (2000) 404:84-87. Arevalo-Martin et al., Therapeutic action of cannabinoids in a murine model of multiple sclerosis J Neurosci. (2003) 23:2511-2516.). Taken together, these results support the notion that CB2 receptor modulators can be employed for the treatment of medical conditions having an inflammatory component.

In addition to inflammation, CB2 agonists have been shown to inhibit pain and emesis. For instance, CB2 selective agonists blunt the pain response induced by thermal or other stimuli (Malan et al., CB2 cannabinoid receptor-mediated peripheral antinociception. Pain. (2001) 93:239-45 and Nackley et al., Selective activation of cannabinoid CB(2) receptors suppresses spinal fos protein expression and pain behavior in a rat model of inflammation. Neuroscience (2003) 119:747-57.) CB2 activation has also been demonstrated to inhibit neuropathic pain response (Ibrahim et al., Activation of CB2 cannabinoid receptors by AM1241 inhibits experimental neuropathic pain: pain inhibition by receptors not present in the CNS. Proc Natl Acad Sci USA. (2003) 100:10529-33.) Finally, in contrast to the earlier data which did not find CB2 in the brain, a recent article demonstrated the expression of CB2 in the brain, at about 1.5% of the level in the spleen. CB2 activation is shown by this article to be responsible for the anti-emetic effect of endocannabinoid (Van Sickle et al., Identification and functional characterization of brainstem cannabinoid CB2 receptors. Science. 2005 310:329-332.) The foregoing results confirm that CB2 agonists can be used for the treatment of inflammatory and neuropathic pain as well as emesis.

### BRIEF SUMMARY OF THE INVENTION

The present invention provides novel compounds which bind to and are agonists, antagonists or inverse agonists of the CB2 receptor. The invention also provides a method and pharmaceutical compositions for treating inflammation by way of the administration of therapeutic amounts of these compounds. Lastly, the invention provides a method and pharmaceutical compositions for treating pain by way of the administration of therapeutic amounts of a subset of the new compounds which are CB2 agonists.

### DETAILED DESCRIPTION OF THE INVENTION

In its broadest generic aspect the invention provides compounds of the formula

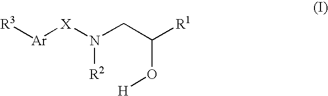

wherein:

R¹ is a hydrogen, C₁-C₆ alkyl, C₃-C₁₀ cycloalkyl, aryl or heteroaryl, wherein the aryl or heteroaryl are each optionally substituted with 1-3 substituents; or, R¹ is C₁-C₃ alkyl substituted with Z-R⁴, wherein Z is O, S, SO₂, NH, NMe or CH₂ and R⁴ is aryl or heteroaryl, wherein the aryl or heteroaryl is optionally substituted with 1-3 substituents;

R² is hydrogen or C₁-C₆ alkyl;

X is a methylene group which is optionally mono- or di-substituted with methyl; or X is a carbonyl group;

Ar is a divalent moiety which is either phenylene or a six-membered heteroarylene, which divalent moiety is optionally mono- or disubstituted with moieties selected from the group consisting of C₁-C₆ alkyl (optionally substituted by 1-3 halogens), C₃-C₁₀ cycloalkyl and halogen; or Ar is a fused aromatic system which can be a 5,6-, or 6,6-bicyclic ring structure and may contain heteroatoms such as O and N;

R³ is H, NR⁵R⁶, OR⁶, SO₂R⁶, or CH₂R⁶, wherein R⁵ is hydrogen or C₁-C₆ alkyl, and R⁶ is aryl or heteroaryl, wherein the aryl or heteroaryl are each optionally substituted with 1-3 substituents.

In a first subgeneric aspect, the invention provides compounds of the formula I wherein,

R¹ is a hydrogen, C₁-C₆ alkyl, C₃-C₁₀ cycloalkyl, phenyl, or pyridyl;

R² is hydrogen or C₁-C₆ alkyl;

X is a methylene group (which is optionally mono- or disubstituted with methyl) or a carbonyl group;

Ar is a divalent moiety which is either phenylene or a six-membered heteroarylene, which divalent moiety is optionally mono- or disubstituted with moieties selected from the group consisting of C₁-C₆ alkyl (optionally substituted by 1-3 halogens), C₃-C₁₀ cycloalkyl and halogen; or Ar is a fused aromatic system which can be a 5,6-, or 6,6-bicyclic ring structure and may contain heteroatoms such as O and N;

R³ is H, NR⁵R⁶, OR⁶, SO₂R⁶, or CH₂R⁶, wherein R⁵ is hydrogen or C₁-C₆ alkyl and R⁶ is an aryl or heteroaryl moiety, wherein the aryl or heteroaryl moiety is optionally substituted with a substituent selected from the group consisting of C₁-C₆ alkyl (which is optionally substituted with 1 to 3 halogen atoms), C₁-C₆ alkoxy (which is optionally substituted with 1 to 3 halogen atoms), C₁-C₆ alkoxycarbonyl, C₁-C₆ alkylaminocarbonyl, C₁-C₆ dialkylaminocarbonyl, hydroxyl, halogen, cyano or nitro.

In a further subgeneric aspect, the invention provides compounds of the formula I wherein,

R¹ is a phenyl;

R² is hydrogen or C₁-C₆ alkyl;

X is a methylene group;

Ar is a 1,4-phenylene or 1,4-pyridylene; or Ar is a quinoline;

R³ is H, NR⁵R⁶, OR⁶, SO₂R⁶, or CH₂R⁶, wherein R⁵ is hydrogen or methyl and R⁶ is a phenyl, wherein the phenyl is optionally mono- or di-substituted with methyl or chlorine or a combination of the two.

The invention also includes tautomers, prodrugs and pharmaceutically acceptable salts of the above-described compounds of formula I. In addition, the invention includes amorphous or crystalline forms of the compounds, and isolated isomorphs or polymorphic mixtures, if present.

Compounds of the formula I are agonists, antagonists or inverse agonists of the CB2 receptor and modulate the activity of this receptor. By virtue of this fact the compounds of the formula I can be used for treating inflammation, in a manner described more fully below.

Those compounds of the formula I which are agonists of the CB2 receptor can additionally be used for treating pain, in a manner described more fully below.

The compounds of formula I may be made using the general synthetic methods described below, which also constitute part of the invention.

### General Synthetic Methods

The invention also provides processes for making compounds of Formula (I). In all schemes, unless specified otherwise, Ar, R¹, R², R³, and X in the formulas below shall have the meaning of Ar, R¹, R², R³, and X in Formula (I) of the invention described herein above. Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization. The examples which follow are illustrative and, as recognized by one skilled in the art, particular reagents or conditions could be modified as needed for individual compounds without undue experimentation. Starting materials and intermediates used, in the schemes below, are either commercially available or easily prepared from commercially available materials by those skilled in the art.

Compounds of Formula (I) may be synthesized by the method illustrated in Scheme 1

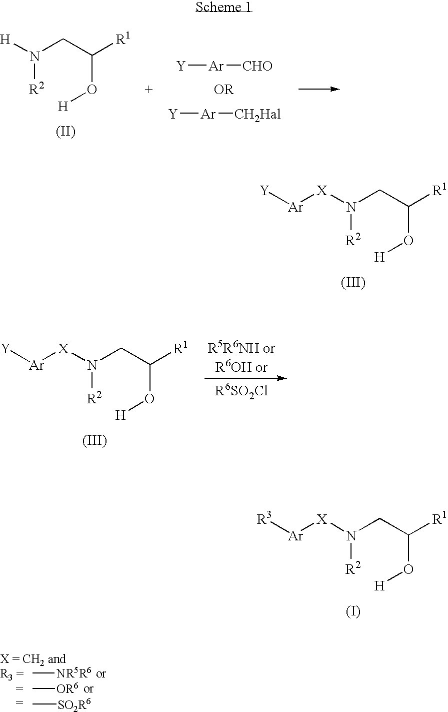

As illustrated in Scheme 1, reacting an amino alcohol starting material of formula II with an aldehyde of formula Y—Ar—CHO (Y is Cl, F, or Br) or a ketone, in a suitable solvent such as THF, in the presence of a suitable reducing agent provides the alkylated amine of formula III.

Alternatively, the starting amino alcohol II may also be reacted with an halide of formula Y—Ar—CH₂-Hal (Hal is Cl, Br or I), in a suitable solvent such as acetonitrile, in the presence of a base such as potassium carbonate to provide the alkylated amine of formula III. The appropriately substituted starting amino alcohol II may be obtained either commercially or made by procedures known to one skilled in the art.

Reacting the intermediate of formula III with an amine of formula R⁵R⁶NH in the presence of a suitable base with or without palladium catalyst provides a compound of formula (I) where R³ is —NR⁵R⁶. Alternatively, reacting the intermediate of formula III with a phenol of formula R⁶OH, in a suitable solvent, in the presence of a suitable base and copper iodide provides a compound of formula (I) where R³ is —OR⁶. The intermediate of formula III may also be reacted with a sulfonyl chloride of formula R⁶SO₂Cl, in a suitable solvent, in the presence of a suitable base to provide a compound of formula (I) where R³ is —SO₂R⁶. The appropriately substituted starting amine, phenol and sulfonyl chloride may be obtained either commercially or made by procedures known to one skilled in the art.

Further modification of the initial product of formula (I), by methods known in the art and illustrated in the Examples below, may be used to prepare additional compounds of this invention.

### EXAMPLES

The manner in which the compounds of the invention can be made will be further understood by way of the following Examples.

### Example 1

### 2-{[4-(2,5-Dimethyl-phenylamino)-benzyl]-methyl-amino}-1-phenyl-ethanol

### 2-[(4-bromo-benzyl)-methyl-amino]-1-phenyl-ethanol

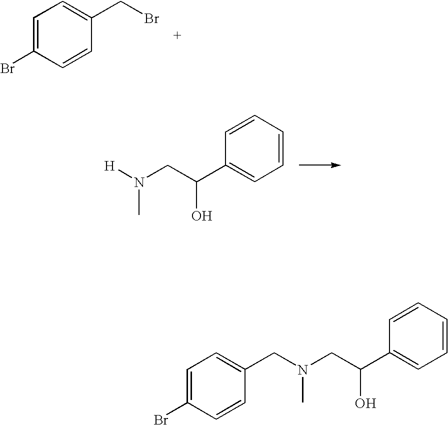

5 g of 4-bromobenzyl bromide was dissolved in acetonitrile and 3.025 g of α-(methylaminomethyl)-benzyl alcohol and 8.295 g of potassium carbonate was added. The mixture was stirred at room temperature overnight, filtered and the cake was washed with more acetonitrile. The filtrate was concentrated to afford 6.418 g of slightly yellow oil. 100% yield. ES MS (+) m/z 320, 322

### 2-{[4-(2,5-Dimethyl-phenylamino)-benzyl]-methyl-amino}-1-phenyl-ethanol

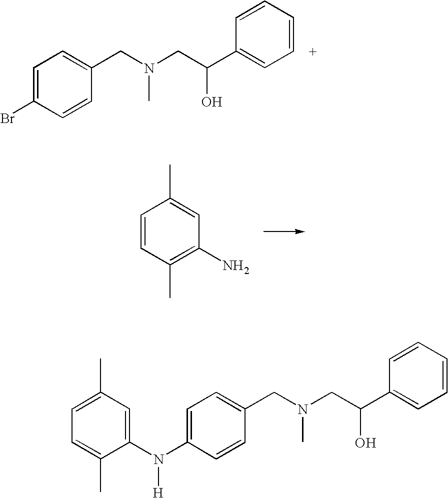

A microwave vessel was charged with 14.3 mg of tris(dibenzylideneacetone)dipallidium (0), 13 mg of 2-(cyclohexylphosphino)biphenyl and 100 mg of 2-[(4-bromo-benzyl)-methyl-amino]-1-phenyl-ethanol. The vessel was evacuated and back-filed with argon three times. 47 μL of 2,5-dimethylaniline and 0.686 mL of 1M lithium bis(trimethylsilyl)amide in THF was then added. The mixture was heated in a microwave reactor at 120 C for 1 hour. The reaction mixture was cooled and filtered through celite, washing with ethyl acetate. The filtrate was concentrated and purified by column chromatography using methanol/dichloromethane as eluent mixtures to afford 26 mg of product. 23% yield. ES MS (+) m/z 361

### Example 2

### 2-[methyl-(4-phenylamino-benzyl)-amino]-1-phenyl-ethanol

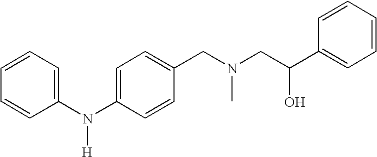

The above compound was made in a similar manner as Example 1 but with the appropriate aniline. 61% yield. ES MS (+) m/z 333

### Example 3

### 2-{[4-(2-chloro-5-methyl-phenylamino)-benzyl]-methyl-amino}-1-phenyl-ethanol

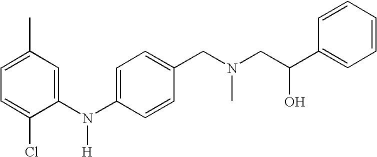

The above compound was made in a similar manner as Example 1 but with the appropriate aniline and purified further by preparatory LC-MS. 29% yield. ES MS (+) m/z 381

### Example 4

### 2-{[4-(2-chloro-phenylamino)-benzyl]-methyl-amino}-1-phenyl-ethanol

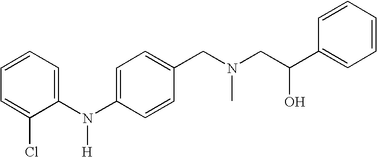

The above compound was made in a similar manner as Example 1 but with the appropriate aniline and purified further by preparatory LC-MS. 23% yield. ES MS (+) m/z 367

### Example 5

### (R)-2-{[4-(2-chloro-phenylamino)-benzyl]-methyl-amino}-1-phenyl-ethanol

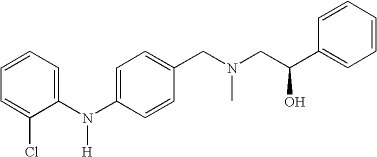

The above compound was made in a similar manner as Example 1 but with the appropriate aniline and chiral arylhalide and purified further by preparatory LC-MS. 31% yield. ES MS (+) m/z 367

### Example 6

### 2-[methyl-4(o-tolylamino-benzyl)-amino]-1-phenyl-ethanol

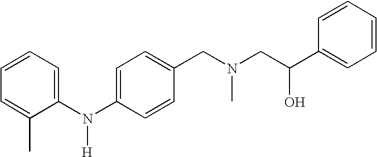

The above compound was made in a similar manner as Example 1 but with the appropriate aniline and purified further by preparatory LC-MS. 11% yield. ES MS (+) m/z 347

### Example 7

### 2-{[6-(2,5-dimethyl-phenylamino)-pyridin-3-ylmethyl]-methyl-amino}-1-phenyl-ethanol

### 2-[(6-Bromo-pyridin-3-ylmethyl)-methyl-amino]-1-phenyl-ethanol

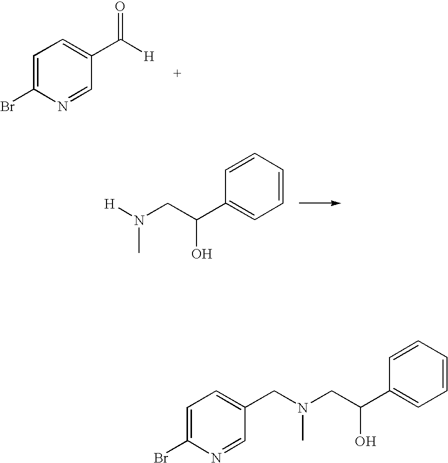

To a solution of 813 mg of α-(methylaminomethyl)-benzyl alcohol in 20 mL THF was added 0.77 mL of acetic acid and 1 g of 6-bromo-3-pyridinecarboxaldehyde. The mixture was stirred at room temperature for 20 minutes and then added 2.28 g of sodium triacetoxyborohydride. The reaction mixture was stirred at room temperature overnight. The mixture was quenched by saturated sodium bicarbonate aqueous solution and extracted with ethyl acetate three times. The organic layer was washed with brine and dried with sodium sulfate. The filtrate was concentrated and purified by flash chromatography using methanol/dichloromethane as eluent mixtures to afford 1.29 g of product. 75% yield. ES MS (+) m/z 321, 323

### 2-{[6-(2,5-dimethyl-phenylamino)-pyridin-3-ylmethyl]-methyl-amino}-1-phenyl-ethanol

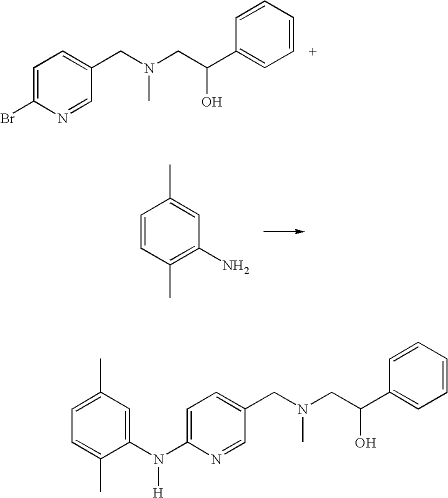

A microwave vessel was charged with 23 mg of tris(dibenzylideneacetone)dipalladium (0) and 200 mg of sodium tert-butoxide. The vessel was evacuated and back-filled with argon three times. Then added 34 mg of 2,8,9-triisobutyl-2,5,8,9-tetraaza-1-phosphabicyclo[3,3,3]undecane in 1 mL of toluene, 200 mg of 2-[(6-Bromo-pyridin-3-ylmethyl)-methyl-amino]-1-phenyl-ethanol in 2 mL of toluene and 116 μL of 2,5-dimethylaniline. The mixture was heated in a microwave reactor at 120° C. for 1 hour. The reaction mixture was cooled and filtered through celite, washing with ethyl acetate. The filtrate was concentrated and purified by flash chromatography using methanol/dichloromethane as eluent mixtures. It was further purified by preparatory thin layer chromatography using 10% methanol/dichloromethane as solvent mixtures to afford 34 mg of product. 15% yield. ES MS (+) m/z 362

### Example 8

### 2-{[6-(2-chloro-5-methyl-phenylamino)-pyridin-3-ylmethyl]-methyl-amino}-1-phenyl-ethanol

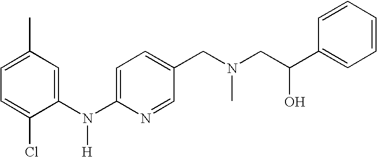

The above compound was made in a similar manner as Example 7 but with the appropriate aniline. 28% yield. ES MS (+) m/z 382

### Example 9

### 2-{[6-(5-chloro-2-methyl-phenylamino)-pyridin-3-ylmethyl]-methyl-amino}-1-phenyl-ethanol

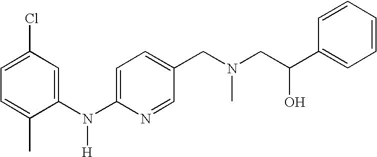

The above compound was made in a similar manner as Example 7 but with the appropriate aniline and purified further by preparatory LC-MS. 10% yield. ES MS (+) m/z 382

### Example 10

### 2-{[6-(2-chloro-phenylamino)-pyridin-3-ylmethyl]-methyl-amino}-1-phenyl-ethanol

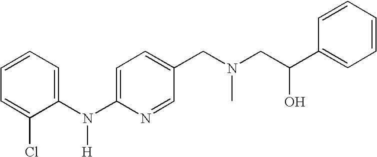

The above compound was made in a similar manner as Example 7 but with the appropriate aniline. The compound was further purified preparatory thin layer chromatography using ethyl acetate/hexane as solvent mixtures. 20% yield. ES MS (+) m/z 368

### Example 11

### 2-[methyl-(6-o-tolylamino-pyridin-3-ylmethyl)-amino]-1-phenyl-ethanol

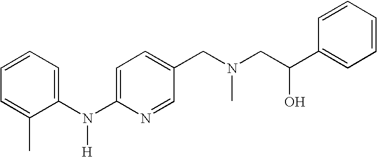

The above compound was made in a similar manner as Example 7 but with the appropriate aniline. The compound was further purified preparatory thin layer chromatography using ethyl acetate/hexane as solvent mixtures. 15% yield. ES MS (+) m/z 348

### Example 12

### (S)-2-(4-Benzenesulfonyl-benzylamino)-1-phenyl-ethanol

### 4-Benzenesulfonyl-benzaldehyde

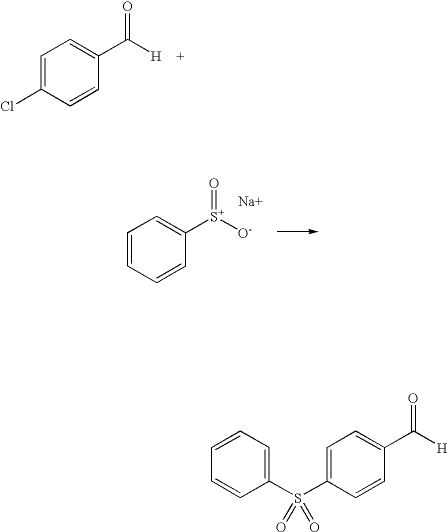

A microwave vessel was charged with 1 g of 4-chlorobenzaldehyde in 6 mL of DMSO and 1.75 g of sodium benzenesulfinate. The vessel was sealed and heated in a microwave reactor at 180° C. for 1.5 hours. The mixture was cooled and poured into 12 mL of ice water. Filtered and the solid was purified by flash chromatography using ethyl acetate/hexane as eluent mixtures to afford 1.34 g of product. 76% yield.

### (S)-2-(4-Benzenesulfonyl-benzylamino)-1-phenyl-ethanol

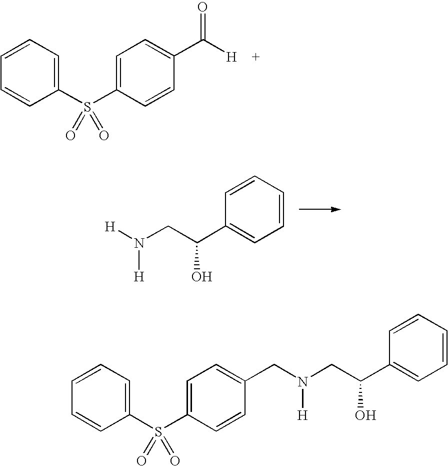

To a solution of 111 mg of (S)-(+)-2-amino-1-phenylethanol in 10 mL of THF was added 116 μL of acetic acid and 200 mg of 4-benzenesulfonyl-benzaldehyde. The mixture was stirred at room temperature for 20 minutes and then added 344 mg of sodium triacetoxyborohydride. The reaction mixture was stirred at room temperature overnight. The mixture was quenched with saturated sodium carbonate aqueous solution and extracted with ethyl acetate three times. The organic layer was washed with brine and dried with magnesium sulfate. The filtrate was concentrated and purified by flash chromatography using methanol/dichloromethane as eluent mixtures to afford 202 mg of product. 68% yield. ES MS (+) m/z 368

### Example 13

### (R)-2-(4-Benzenesulfonyl-benzylamino)-1-phenyl-ethanol

### (R)-2-(4-Benzenesulfonyl-benzylamino)-1-phenyl-ethanol

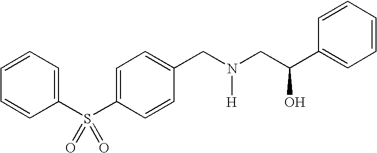

The above compound was made in a similar manner as (S)-2-(4-Benzenesulfonyl-benzylamino)-1-phenyl-ethanol in Example 12 but with the appropriate chiral amine. The product was trituated in ether to afford white solid. 84% yield. ES MS (+) m/z 369

### Example 14

### (S)-2-[Methyl-(4-phenylamino-benzyl)-amino]-1-phenyl-ethanol

### (S)-2-(4-Bromo-benzylamino)-1-phenyl-ethanol

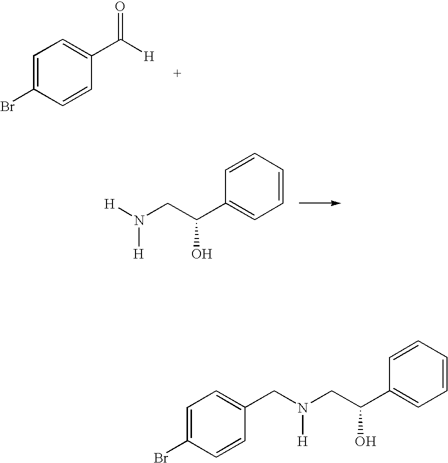

To a solution of 741 mg of (S)-(+)-2-amino-1-phenylethanol in 40 mL of THF was added 774 μL of acetic acid and 1 g of 4-bromobenzaldehyde. The mixture was stirred at room temperature for 20 minutes and then added 2.29 g of sodium triacetoxyborohydride. The reaction mixture was stirred at room temperature for 2 hours. The mixture was quenched with saturated sodium bicarbonate aqueous solution and extracted with ethyl acetate 3 times. The organic layer was washed with brine and dried with magnesium sulfate. The filtrate was concentrated and purified by flash chromatography using methanol/dichloromethane as eluent mixtures to afford 1.22 g of product. 74% yield. ES MS (+) m/z 306, 308

### (S)-2-[(4-Bromo-benzyl)-methyl-amino]-1-phenyl-ethanol

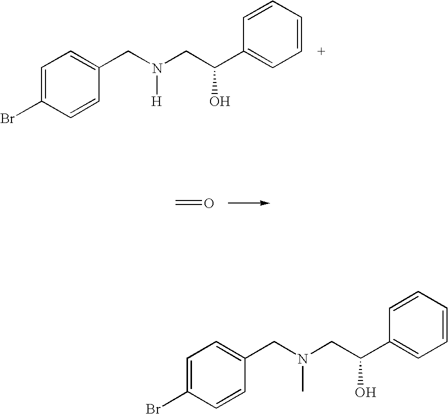

To a solution of 1.19 g of (S)-2-(4-Bromo-benzylamino)-1-phenyl-ethanol in 10 mL of THF was added 558 μL of acetic acid and 2.9 mL of 37% of formaldehyde in water. The mixture was stirred at room temperature for 3.5 hours and then added 1.65 g of sodium triacetoxyborohydride. The reaction mixture was stirred at room temperature overnight. The mixture was quenched with saturated sodium carbonate aqueous solution and extracted with ethyl acetate 3 times. The organic layer was washed with brine and dried with sodium sulfate. The filtrate was concentrated to afford 1.29 g of product. The product was used crude. ES MS (+) m/z 320, 322

### (S)-2-[Methyl-(4-phenylamino-benzyl)-amino]-1-phenyl-ethanol

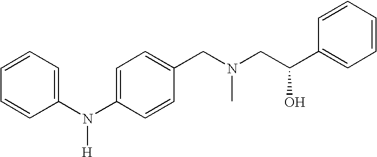

The above compound was made in a similar manner as Example 2 but with the appropriate chiral bromide and purified further by preparatory LC-MS. 38% yield. ES MS (+) m/z 334

### Example 15

### (R)-2-[Methyl-(4-phenylamino-benzyl)-amino]-1-phenyl-ethanol

### (R)-2-[(4-Bromo-benzyl)-methyl-amino]-1-phenyl-ethanol

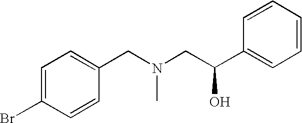

The above compound was made in a similar manner as (S)-2-(4-Bromo-benzylamino)-1-phenyl-ethanol in Example 14 but with the appropriate amine. The product was further purified by preparatory thin layer chromatography using 50% of ethyl acetate/hexane as solvent mixtures. 62% yield. ES MS (+) m/z 320, 322

### (R)-2-[Methyl-(4-phenylamino-benzyl)-amino]-1-phenyl-ethanol

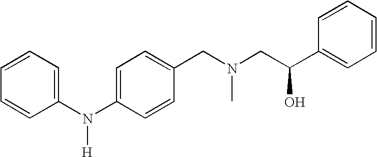

The above compound was made in a similar manner as Example 2 but with the appropriate chiral bromide and purified further by preparatory LC-MS. 75% yield. ES MS (+) m/z 334

### Example 16

### (R)-1-Phenyl-2-[(quinolin-3-ylmethyl)-amino]-ethanol

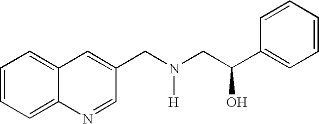

The above compound was made in a similar manner as (S)-2-(4-Bromo-benzylamino)-1-phenyl-ethanol in Example 14 but with the appropriate aldehyde and chiral amine. The product was trituated in ether after workup to afford off white solid. 75% yield. ES MS (+) m/z 279

### Example 17

### (R)-2-[(4-Benzenesulfonyl-benzyl)-methyl-amino]-1-phenyl-ethanol

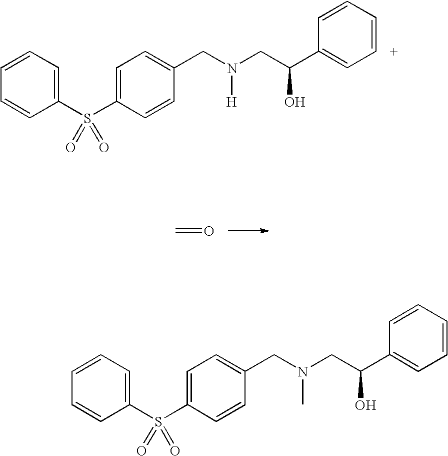

To a solution of 150 mg of (R)-2-(4-Benzenesulfonyl-benzylamino)-1-phenyl-ethanol in 10 mL of THF was added 58 L of acetic acid and 61 L of 37% of formaldehyde in water. The mixture was stirred at room temperature for 20 minutes and then added 173 mg of sodium triacetoxyborohydride. The reaction mixture was stirred at room temperature overnight. The mixture was quenched with saturated sodium carbonate aqueous solution and extracted with dichloromethane three times. The organic layer was washed with brine and dried with sodium sulfate. The filtrate was concentrated and purified by flash chromatography using methanol/dichloromethane as eluent mixtures. The product was further purified by pre-TLC using 50% ethyl acetate/hexane as solvent mixtures to afford product. 69% yield. ES MS (+) m/z 382

### Example 18

### (S)-2-[Methyl-(4-phenoxy-benzyl)-amino]-1-phenyl-ethanol

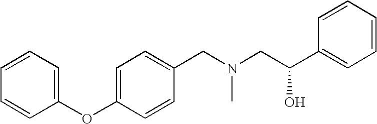

The above compound was made in a similar manner as (S)-2-(4-Bromo-benzylamino)-1-phenyl-ethanol in Example 14 but with the appropriate aldehyde and chiral amine. 87% yield. ES MS (+) m/z 334

### Example 19

### (R)-2-[Methyl-(4-phenoxy-benzyl)-amino]-1-phenyl-ethanol

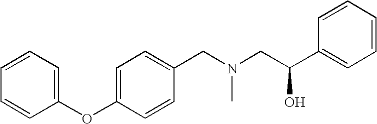

The above compound was made in a similar manner as (S)-2-(4-Bromo-benzylamino)-1-phenyl-ethanol in Example 14 but with the appropriate aldehyde and chiral amine. 82% yield. ES MS (+) m/z 334

### Example 20

### (R)-2-(Methyl-quinolin-3-ylmethyl-amino)-1-phenyl-ethanol

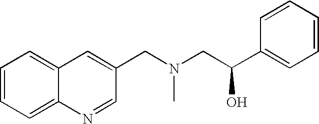

The above compound was made in a similar manner as (S)-2-(4-Bromo-benzylamino)-1-phenyl-ethanol in Example 14 but with the appropriate chiral amine. 37% yield. ES MS (+) m/z 293

### Example 21

### (S)-2-[Methyl-(6-phenoxy-pyridin-3-ylmethyl)-amino]-1-phenyl-ethanol

### (S)-2-[(6-Phenoxy-pyridin-3-ylmethyl)-amino]-1-phenyl-ethanol

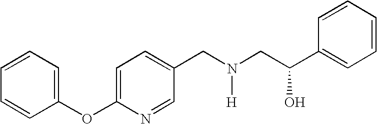

The above compound was made in a similar manner as (S)-2-(4-Bromo-benzylamino)-1-phenyl-ethanol in Example 14 but with the appropriate aldehyde and chiral amine. The product was trituated in ether after workup to afford off white solid. 42% yield. ES MS (+) m/z 321

### (S)-2-[Methyl-(6-phenoxy-pyridin-3-ylmethyl)-amino]-1-phenyl-ethanol

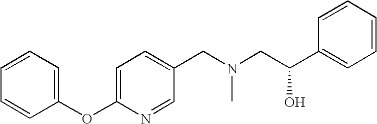

The above compound was made in a similar manner as Example 14 but with the appropriate chiral amine. 50% yield. ES MS (+) m/z 335

### Example 22

### (S)-1-Phenyl-2-[(quinolin-3-ylmethyl)-amino]-ethanol

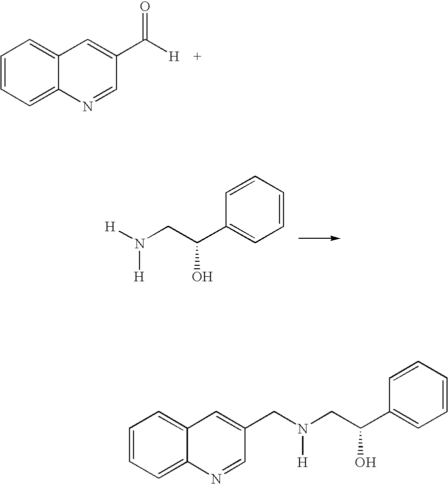

The above compound was made in a similar manner as (S)-2-(4-Bromo-benzylamino)-1-phenyl-ethanol in Example 14 but with the appropriate aldehyde and chiral amine. 83% yield. ES MS (+) m/z 279

### Assessment of Biological Properties

The biological properties of the compounds of the formula I were assessed using the assays described below.

### A. Human CB1 and CB2 Receptor Binding:

### Experimental Method:

CB2 membranes were purchased and made from HEK293 EBNA cells stably transfected with human CB2 receptor cDNA (Perkin Elmer Life and Analytical Sciences). CB1 membranes were isolated from HEK cells stably co-transfected with human CB1 receptor and Gα16 cDNA's. The membrane preparation was bound to scintillation beads (Ysi-Poly-L-lysine SPA beads, GE Healthcare) for 4 hours at room temperature in assay buffer containing 50 mM Tris, pH 7.5, 2.5 mM EDTA, 5 mM MgCl₂, 0.8% fatty acid free Bovine Serum Albumin. Unbound membrane was removed by washing in assay buffer. Membrane-bead mixture was added to 96-well assay plates in the amounts of 1 μg membrane per well (CB2) or 2.5 ug per well (CB1) and 1 mg SPA bead per well. Compounds were added to the membrane-bead mixture in dose-response concentrations ranging from 1×10⁻⁵ M to 1×10⁻¹⁰ M with 0.25% DMSO, final. The competition reaction was initiated with the addition of ³H-CP55940 (Perkin Elmer Life and Analytical Sciences) at a final concentration of 1.5 nM (CB2) or 2.5 nM (CB1). The reaction was incubated at room temperature for 18 hours and read on TopCount NXT plate reader. Total and non-specific binding was determined in the absence and presence of 1.25 uM Win 55212 (Sigma). IC50 values for each compound were calculated as the concentration of compound that inhibits the specific binding of the radioactively labeled ligand to the receptor by 50% using the XLFit 4.1 four parameter logistic model. IC50 values were converted to inhibition constant (Ki) values using Cheng-Prusoff equation.

B. CB2R Mediated Modulation of cAMP Synthesis:

Compounds of the invention were evaluated for their CB2 agonist or inverse agonistic activity in accordance with the following experimental method. Compounds which were shown to bind to CB2 by the binding assay described above but which were not shown to exhibit CB2R-mediated modulation of cAMP synthesis by this assay were presumed to be CB2 antagonists.

### Experimental Method:

CHO cells expressing human CB2R (Euroscreen) were plated at a density of 5000 cells per well in 384 well plates and incubated overnight at 37° C. After removing the media, the cells were treated with test compounds diluted in stimulation buffer containing 1 mM IBMX, 0.25% BSA and 10 uM Forskolin. The assay was incubated for 30 minutes at 37° C. Cells were lysed and the cAMP concentration was measured using DiscoverX-XS cAMP kit, following the manufacturer's protocol. In this setting, agonists will decrease forskolin induced production of cAMP while inverse agonists will further increase forskolin induced production of cAMP. EC50 of agonists were calculated as follows. The maximal amount of cAMP produced by forskolin compared to the level of cAMP inhibited by 1 uM CP55940 is defined as 100%. The EC50 value of each test compound was determined as the concentration at which 50% of the forskolin-stimulated cAMP synthesis was inhibited. Data was analyzed using a four-parameter logistic model. (Model 205 of XLfit 4.0).

C. CB1R Mediated Modulation of cAMP Synthesis:

Compounds of the invention were evaluated for their CB1 agonist or inverse agonistic activity in accordance with the following experimental method. Compounds which were shown to bind to CB1 by the binding assay described above but which were not shown to exhibit CB1R-mediated modulation of cAMP synthesis by this assay were presumed to be CB1 antagonists.

### Experimental Method:

CHO cells expressing human CB1R (Euroscreen) were plated at a density of 5000 cells per well in 384 well plates and incubated overnight at 37° C. After removing the media, the cells were treated with test compounds diluted in stimulation buffer containing 1 mM IBMX, 0.25% BSA and 10 uM Forskolin. The assay was incubated for 30 minutes at 37° C. Cells were lysed and the cAMP concentration was measured using DiscoverX-XS cAMP kit, following the manufacturer's protocol. In this setting, agonists will decrease forskolin induced production of cAMP while inverse agonists will further increase forskolin induced production of cAMP. EC50 of agonists were calculated as follows. The maximal amount of cAMP produced by forskolin compared to the level of cAMP inhibited by 1 uM CP55940 is defined as 100%. The EC50 value of each test compound was determined as the concentration at which 50% of the forskolin-stimulated cAMP synthesis was inhibited. Data was analyzed using a four-parameter logistic model. (Model 205 of XLfit 4.0).

### Compounds Having Agonist Activity

Through the use of the above described assays the following compounds were found to exhibit agonistic activity and thus to be particularly well suited for the treatment of pain as well as for the treatment of inflammation.
(R)-2-{[4-(2-Chloro-phenylamino)-benzyl]-methyl-amino}-1-phenyl-ethanol;
2-{[6-(2-Chloro-phenylamino)-pyridin-3-ylmethyl]-methyl-amino}-1-phenyl-ethanol;
2-[Methyl-(4-o-tolylamino-benzyl)-amino]-1-phenyl-ethanol;
2-[Methyl-(6-o-tolylamino-pyridin-3-ylmethyl)-amino]-1-phenyl-ethanol;
2-{[4-(2-Chloro-phenylamino)-benzyl]-methyl-amino}-1-phenyl-ethanol;
(R)-2-[(4-Benzenesulfonyl-benzyl)-methyl-amino]-1-phenyl-ethanol;
(R)-2-[Methyl-(4-phenylamino-benzyl)-amino]-1-phenyl-ethanol;
2-[Methyl-(4-phenylamino-benzyl)-amino]-1-phenyl-ethanol;
2-{[6-(5-Chloro-2-methyl-phenylamino)-pyridin-3-ylmethyl]-methyl-amino}-1-phenyl-ethanol;
(R)-2-[Methyl-(4-phenoxy-benzyl)-amino]-1-phenyl-ethanol;
2-{[6-(2-Chloro-5-methyl-phenylamino)-pyridin-3-ylmethyl]-methyl-amino}-1-phenyl-ethanol;
2-{[6-(2,5-Dimethyl-phenylamino)-pyridin-3-ylmethyl]-methyl-amino}-1-phenyl-ethanol;
(R)-2-(methyl-quinolin-3-ylmethyl-amino)-1-phenyl-ethanol;
2-{[4-(2,5-Dimethyl-phenylamino)-benzyl]-methyl-amino}-1-phenyl-ethanol;
(S)-2-[Methyl-(6-phenoxy-pyridin-3-ylmethyl)-amino]-1-phenyl-ethanol;
2-{[4-(2-chloro-5-methyl-phenylamino)-benzyl]-methyl-amino}-1-phenyl-ethanol;
(S)-1-Phenyl-2-[(quinolin-3-ylmethyl)-amino]-ethanol

Of the above compounds, the following are preferred:
(R)-2-{[4-(2-Chloro-phenylamino)-benzyl]-methyl-amino}-1-phenyl-ethanol;
2-{[6-(2-Chloro-phenylamino)-pyridin-3-ylmethyl]-methyl-amino}-1-phenyl-ethanol;
2-[Methyl-(4-o-tolylamino-benzyl)-amino]-1-phenyl-ethanol;
2-[Methyl-(6-o-tolylamino-pyridin-3-ylmethyl)-amino]-1-phenyl-ethanol;
2-{[4-(2-Chloro-phenylamino)-benzyl]-methyl-amino}-1-phenyl-ethanol;
(R)-2-[(4-Benzenesulfonyl-benzyl)-methyl-amino]-1-phenyl-ethanol;
(R)-2-[Methyl-(4-phenylamino-benzyl)-amino]-1-phenyl-ethanol;
2-[Methyl-(4-phenylamino-benzyl)-amino]-1-phenyl-ethanol;
2-{[6-(5-Chloro-2-methyl-phenylamino)-pyridin-3-ylmethyl]-methyl-amino}-1-phenyl-ethanol;
(R)-2-[Methyl-(4-phenoxy-benzyl)-amino]-1-phenyl-ethanol;
2-{[6-(2-Chloro-5-methyl-phenylamino)-pyridin-3-ylmethyl]-methyl-amino}-1-phenyl-ethanol;
2-{[6-(2,5-Dimethyl-phenylamino)-pyridin-3-ylmethyl]-methyl-amino}-1 phenyl-ethanol.

### Therapeutic Use

As can be demonstrated by the assays described above, the compounds of the invention are useful in modulating the CB2 receptor function. By virtue of this fact, these compounds have therapeutic use in treating disease-states and conditions mediated by the CB2 receptor function or that would benefit from modulation of the CB2 receptor function.

As the compounds of the invention modulate the CB2 receptor function, they have very useful anti-inflammatory and immune-suppressive activity and they can be used in patients as drugs, particularly in the form of pharmaceutical compositions as set forth below, for the treatment of disease-states and conditions.

As noted before, those compounds which are CB2 agonists can also be employed for the treatment of pain.

The agonist, antagonist and inverse agonist compounds according to the invention can be used in patients as drugs for the treatment of the following disease-states or indications that are accompanied by inflammatory processes:
(i) Lung diseases: e.g. asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;
(ii) Rheumatic diseases or autoimmune diseases or musculoskeletal diseases: all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis; collagenoses of any genesis, e.g., systemic lupus erythematosus, scleroderma, polymyositis, dermatomyositis, Sjögren syndrome, Still disease, Felty syndrome; and osteoporosis and other bone resorption diseases;
(iii) Allergic diseases: all forms of allergic reactions, e.g., angioneurotic edema, hay fever, insect bites, allergic reactions to drugs, blood derivatives, contrast agents, etc., anaphylactic shock (anaphylaxis), urticaria, angioneurotic edema, and contact dermatitis;
(iv) Vascular diseases: panarteritis nodosa, polyarteritis nodosa, periarteritis nodosa, arteritis temporalis, Wegner granulomatosis, giant cell arthritis, atherosclerosis, reperfusion injury and erythema nodosum;
(v) Dermatological diseases: e.g. dermatitis, psoriasis; sunburn, burns, eczema;
(vi) Renal diseases: e.g. nephrotic syndrome; and all types of nephritis, e.g., glomerulonephritis; pancreatits;
(vii) Hepatic diseases: e.g. acute liver cell disintegration; acute hepatitis of various genesis, e.g., viral, toxic, drug-induced; and chronically aggressive and/or chronically intermittent hepatitis;
(viii) Gastrointestinal diseases: e.g. inflammatory bowel diseases, irritable bowel syndrome, regional enteritis (Crohns disease), colitis ulcerosa; gastritis; aphthous ulcer, celiac disease, regional ileitis, gastroesophageal reflux disease;
(ix) Neuroprotection: e.g. in the treatment of neurodegeneration following stroke; cardiac arrest; pulmonary bypass; traumatic brain injury; spinal cord injury or the like;
(x) Eye diseases: allergic keratitis, uveitis, or iritis; conjunctivitis; blepharitis; neuritis nervi optici; choroiditis; glaucoma and sympathetic ophthalmia;
(xi) Diseases of the ear, nose, and throat (ENT) area: e.g. tinnitus; allergic rhinitis or hay fever; otitis extema; caused by contact eczema, infection, etc.; and otitis media;
(xii) Neurological diseases: e.g. brain edema, particularly tumor-related brain edema; multiple sclerosis; acute encephalomyelitis; meningitis; acute spinal cord injury; trauma; dementia, particularly degenerative dementia (including senile dementia, Alzheimer's disease; Parkinson's disease and Creutzfeldt-Jacob disease; Huntington's chorea, Pick's disease; motor neuron disease), vascular dementia (including multi-infarct dementia) as well as dementia associated with intracranial space occupying lesions; infections and related conditions (including HIV infection); Guillain-Barre syndrome; myasthenia gravis, stroke; and various forms of seizures, e.g., nodding spasms;
(xiii) Blood diseases: acquired hemolytic anemia; aplastic anemia, and idiopathic thrombocytopenia;
(xiv) Tumor diseases: acute lymphatic leukemia; Hodgkin's disease, malignant lymphoma; lymphogranulomatoses; lymphosarcoma; solid malignant tumors; extensive metastases,;
(xv) Endocrine diseases: endocrine opthalmopathy; endocrine orbitopathia; thyrotoxic crisis; Thyroiditis de Quervain; Hashimoto thyroiditis; Morbus Basedow; granulomatous thyroiditis; struma lymphomatosa; and Graves disease; type I diabetes (insulin-dependent diabetes);
(xvi) Organ and tissue transplantations and graft-versus-host diseases;
(xvii) Severe states of shock, e.g., septic shock, anaphylactic shock, and systemic inflammatory response syndrome (SIRS);
(xviii) Acute pain such as dental pain, perioperative, post-operative pain, traumatic pain, muscle pain, pain in burned skin, sun burn, trigeminal neuralgia, sun burn; spasm of the gastrointestinal tract or uterus, colics;
(xix) Visceral pain such as pain associated with chronic pelvic pain, pancreatitis, peptic ulcer, interstitial cystitis, renal colic, angina, dysmenorrhoea, menstruation, gynaecological pain, irritable bowel syndrome (IBS), non-ulcer dyspepsia, non-cardiac chest pain, myocardial ischemia;
(xx) Neuropathic pain such as low back pain, non-herpetic neuralgia, post herpetic neuralgia, diabetic neuropathy, nerve injury, acquired immune deficiency syndrome (AIDS) related neuropathic pain, head trauma, painful traumatic mononeuropathy, toxin and chemotherapy induced pain, phantom limb pain, painful polyneuropathy, thalamic pain syndrome, post-stroke pain, central nervous system injury, post surgical pain, stump pain, repetitive motion pain, pain induced by post mastectomy syndrome, multiple sclerosis, root avulsions, postthoracotomy syndrome, neuropathic pain associated hyperalgesia and allodynia.
(xxi) Inflammatory/nociceptive pain induced by or associated with disorders such as osteoarthritis, rheumatoid arthritis, rheumatic disease, teno-synovitis, gout, vulvodynia, myofascial pain (muscular injury, fibromyalgia), tendonitis, osteoarthritis, juvenile arthritis, spondylitis, gouty arthritis, psoriatic arthritis, muscoskeletal pain, fibromyalgia, sprains and strains, sympathetically maintained pain, myositis, pain associated with migraine, toothache, influenza and other viral infections such as the common cold, rheumatic fever, systemic lupus erythematosus;
(xxii) Cancer pain induced by or associated with tumors such as lymphatic leukemia; Hodgkin's disease, malignant lymphoma; lymphogranulomatoses; lymphosarcoma; solid malignant tumors; extensive metastases;
(xxiii) Headache such as cluster headache, migraine with and without aura, tension type headache, headache with different origins, headache disorders including prophylactic and acute use;
(xxiv) various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis, pyrexia. edema resulting from trauma associated with burns, sprains or fracture, cerebral oedema and angioedema, Diabetes such as diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion).
Other indications include: epilepsy, septic shock e.g. as antihypovolemic and/or antihypotensive agents, cancer, sepsis, osteoporosis, benign prostatic hyperplasia and hyperactive bladder, pruritis, vitiligo, general gastrointestinal disorders, disturbances of visceral motility at respiratory, genitourinary, gastrointestinal or vascular regions, wounds, burns, tissue damage and postoperative fever, syndromes associated with Itching.
Besides being useful for human treatment, these compounds are also useful for veterinary treatment of companion animals, exotic animals and farm animals, including mammals, rodents, and the like.

For treatment of the above-described diseases and conditions, a therapeutically effective dose will generally be in the range from about 0.01 mg to about 100 mg/kg of body weight per dosage of a compound of the invention; preferably, from about 0.1 mg to about 20 mg/kg of body weight per dosage. For example, for administration to a 70 kg person, the dosage range would be from about 0.7 mg to about 7000 mg per dosage of a compound of the invention, preferably from about 7.0 mg to about 1400 mg per dosage. Some degree of routine dose optimization may be required to determine an optimal dosing level and pattern. The active ingredient may be administered from 1 to 6 times a day.

### Combination Therapy

These compounds may also be employed in combination therapies with the following compounds to treat the above mentioned diseases:

non-steroidal antiinfiammatory drugs (NSAIDs) including COX-2 inhibitors such as propionic acid derivatives (acetaminophen, alminoprofen, benoxaprofen, bucloxic acid, carprofen, fenhufen, fenoprofen, flurbiprofen, fluriprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefenamic acid, and tolfenamic acid), biphenyl-carboxylic acid derivatives, oxicams (isoxicam, meloxicam, piroxicam, sudoxicam and tenoxicam), salicylates (aspirin, acetyl salicylic acid, sulfasalazine choline magnesium salicylate, sodium salicylate, magnesium salicylate, choline salicylate,) and the pyrazolones (apazone, bezpiperylon, feprazone, mofebutazone, oxyphenbutazone, phenylbutazone), and the coxibs (celecoxib, valecoxib, rofecoxib and etoricoxib); diflunisal, etodolac, ketorolac tromethanime, meclofenamate sodium, nabumetone, lomoxicam, nimesulide, remifenzone, salsalate, flosulide, and the like;
glucocorticosteroids such as betamethasone, budesonide, cortisone, dexamethasone, hydrocortisone, methylprednisolone, prednisolone, prednisone, triamcinolone and deflazacort;
immunosuppressive, immunomodulatory, or cytsostatic drugs including but not limited to hydroxychlorquine, D-penicillamine, sulfasalizine, auranofin, gold mercaptopurine, tacrolimus, sirolimus, mycophenolate mofetil, cyclosporine, leflunomide, methotrexate, azathioprine, cyclophosphamide and glatiramer acetate and novantrone, fingolimod (FTY720), minocycline and thalidomide;
anti-TNF antibodies or TNF-receptor antagonists such as but not limited to Etanercept, Infliximab, Adalimumab (D2E7), CDP 571, and Ro 45-2081 (Lenercept), or biologic agents directed against targets such as but not limited to CD-4, CTLA-4, LFA-1, IL-6, ICAM-1, C5 and Natalizumab,
IL-1 receptor antagonists such as but not limited to Kineret; interferon-beta, 1a or 1b including but not limited to Betaseron, Avonex and Rebif, interferon-alpha;

angiogenesis inhibitors such as but not limited to compounds directed against VEGF, taxol, pentoxyfylline

opiate receptor agonists such as morphine, propoxyphene (Darvonu), tramadol, buprenorphin; sodium channel blockers such as carbamazepine, mexiletine, lamotrigine, pregabaline, tectin, NW-1029, CGX-1002;

N-type calcium channel blockers such as Ziconotide, NMED-160, SPI-860; serotonergic and noradrenergic modulators such as SR-57746, paroxetine, duloxetine, clonidine, amitriptyline, citalopram;
histamine H1 receptor antagonists such as bromopheniramine, chlorpheniramine, dexchlorpheniramine, triprolidine, clemastine, diphenhydramine, diphenylpyraline, tripelennamine, hydroxyzine, methdiazine, promethazine, trimeprazine, azatadine, cyproheptadine, antazoline, pheniramine pyrilamine, astemizole, terfenadine, loratadine, cetirizine, desloratadine, fexofenadine and levocetirizine;
histamine H2 receptor antagonists such as cimetidine, famotidine and ranitidine;
proton pump inhibitors such as omeprazole, pantoprazole and esomeprazole; leukotriene antagonists and 5-lipoxygenase inhibitors such as zafirlukast, montelukast, pranlukast and zileuton;
local anesthetics such as ambroxol, lidocaine;
VR1 agonists and antagonists such as NGX-4010, WL-1002, ALGRX-4975, WL-10001, AMG-517;
nicotinic acetylcholine receptor agonists such as ABT-202, A-366833, ABT-594; BTG-102, A-85380, CGX1204;
P2X3 receptor antagonists such as A-317491, ISIS-13920, AZD-9056;
NGF agonists and antagonists such as RI-724, RI-1024, AMG-819, AMG-403, PPH 207;
NK1 and NK2 antagonists such as DA-5018, R-116301; CP-728663, ZD-2249;
NMDA antagonist such as NER-MD-11, CNS-5161, EAA-090, AZ-756, CNP-3381;
potassium channel modulators such as CL-888, ICA-69673, retigabine;
GABA modulators such as lacosamide;
serotonergic and noradrenergic modulators such as SR-57746, paroxetine, duloxetine, clonidine, amitriptyline, citalopram, flibanserin; and
combination with anti-migraine drugs like sumatriptan, zolmitriptan, naratriptan, and eletriptan.

### General Administration and Pharmaceutical Compositions

When used as pharmaceuticals, the compounds of the invention are typically administered in the form of a pharmaceutical composition. Such compositions can be prepared using procedures well known in the pharmaceutical art and comprise at least one compound of the invention. The compounds of the invention may also be administered alone or in combination with adjuvants that enhance stability of the compounds of the invention, facilitate administration of pharmaceutical compositions containing them in certain embodiments, provide increased dissolution or dispersion, increased inhibitory activity, provide adjunct therapy, and the like. The compounds according to the invention may be used on their own or in conjunction with other active substances according to the invention, optionally also in conjunction with other pharmacologically active substances. In general, the compounds of this invention are administered in a therapeutically or pharmaceutically effective amount, but may be administered in lower amounts for diagnostic or other purposes.

Administration of the compounds of the invention, in pure form or in an appropriate pharmaceutical composition, can be carried out using any of the accepted modes of administration of pharmaceutical compositions. Thus, administration can be, for example, orally, buccally (e.g., sublingually), nasally, parenterally, topically, transdermally, vaginally, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, preferably in unit dosage forms suitable for simple administration of precise dosages. The pharmaceutical compositions will generally include a conventional pharmaceutical carrier or excipient and a compound of the invention as the/an active agent, and, in addition, may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration are well-known to those of skill in the art. The state of the art is evidenced, e.g., by Remington: The Science and Practice of Pharmacy, 20th Edition, A. Gennaro (ed.), Lippincott Williams & Wilkins, 2000; Handbook of Pharmaceutical Additives, Michael & Irene Ash (eds.), Gower, 1995; Handbook of Pharmaceutical Excipients, A. H. Kibbe (ed.), American Pharmaceutical Ass'n, 2000; and H. C. Ansel and N. G. Popovish, Pharmaceutical Dosage Forms and Drug Delivery Systems, 5th ed., Lea and Febiger, 1990; each of which is incorporated herein by reference in their entireties to better describe the state of the art.

As one of skill in the art would expect, the forms of the compounds of the invention utilized in a particular pharmaceutical formulation will be selected (e.g., salts) that possess suitable physical characteristics (e.g., water solubility) that is required for the formulation to be efficacious.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration comprise sterile aqueous preparations of a compound of the present invention. These preparations are preferably administered intravenously, although administration can also be effected by means of subcutaneous, intramuscular, or intradermal injection. Injectable pharmaceutical formulations are commonly based upon injectable sterile saline, phosphate-buffered saline, oleaginous suspensions, or other injectable carriers known in the art and are generally rendered sterile and isotonic with the blood. The injectable pharmaceutical formulations may therefore be provided as a sterile injectable solution or suspension in a nontoxic parenterally acceptable diluent or solvent, including 1,3-butanediol, water, Ringer's solution, isotonic sodium chloride solution, fixed oils such as synthetic mono- or diglycerides, fatty acids such as oleic acid, and the like. Such injectable pharmaceutical formulations are formulated according to the known art using suitable dispersing or setting agents and suspending agents. Injectable compositions will generally contain from 0.1 to 5% w/w of a compound of the invention.

Solid dosage forms for oral administration of the compounds include capsules, tablets, pills, powders, and granules. For such oral administration, a pharmaceutically acceptable composition containing a compound(s) of the invention is formed by the incorporation of any of the normally employed excipients, such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such solid pharmaceutical formulations may include formulations, as are well-known in the art, to provide prolonged or sustained delivery of the drug to the gastrointestinal tract by any number of mechanisms, which include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form.

Liquid dosage forms for oral administration of the compounds include emulsions, microemulsions, solutions, suspensions, syrups, and elixirs, optionally containing pharmaceutical adjuvants in a carrier, such as, for example, water, saline, aqueous dextrose, glycerol, ethanol and the like. These compositions can also contain additional adjuvants such as wetting, emulsifying, suspending, sweetening, flavoring, and perfuming agents.

Topical dosage forms of the compounds include ointments, pastes, creams, lotions, gels, powders, solutions, sprays, inhalants, eye ointments, eye or ear drops, impregnated dressings and aerosols, and may contain appropriate conventional additives such as preservatives, solvents to assist drug penetration and emollients in ointments and creams. Topical application may be once or more than once per day depending upon the usual medical considerations. Furthermore, preferred compounds for the present invention can be administered in intranasal form via topical use of suitable intranasal vehicles. The formulations may also contain compatible conventional carriers, such as cream or ointment bases and ethanol or oleyl alcohol for lotions. Such carriers may be present as from about 1% up to about 98% of the formulation, more usually they will form up to about 80% of the formulation.

Transdermal administration is also possible. Pharmaceutical compositions suitable for transdermal administration can be presented as discrete patches adapted to remain in intimate contact with the epidermis of the recipient for a prolonged period of time. To be administered in the form of a transdermal delivery system, the dosage administration will, of course, be continuous rather than intermittent throughout the dosage regimen. Such patches suitably contain a compound of the invention in an optionally buffered, aqueous solution, dissolved and/or dispersed in an adhesive, or dispersed in a polymer. A suitable concentration of the active compound is about 1% to 35%, preferably about 3% to 15%.

For administration by inhalation, the compounds of the invention are conveniently delivered in the form of an aerosol spray from a pump spray device not requiring a propellant gas or from a pressurized pack or a nebulizer with the use of a suitable propellant, e.g., dichlorodifluoromethane, trichlorofluoromethane, dichlorotetrafluoroethane, tetrafluoroethane, heptafluoropropane, carbon dioxide, or other suitable gas. In any case, the aerosol spray dosage unit may be determined by providing a valve to deliver a metered amount so that the resulting metered dose inhaler (MDI) is used to administer the compounds of the invention in a reproducible and controlled way. Such inhaler, nebulizer, or atomizer devices are known in the prior art, for example, in PCT International Publication Nos. WO 97/12687 (particularly FIG. 6 thereof, which is the basis for the commercial RESPIMAT® nebulizer); WO 94/07607; WO 97/12683; and WO 97/20590, to which reference is hereby made and each of which is incorporated herein by reference in their entireties.

Rectal administration can be effected utilizing unit dose suppositories in which the compound is admixed with low-melting water-soluble or insoluble solids such as fats, cocoa butter, glycerinated gelatin, hydrogenated vegetable oils, mixtures of polyethylene glycols of various molecular weights, or fatty acid esters of polyethylene glycols, or the like. The active compound is usually a minor component, often from about 0.05 to 10% by weight, with the remainder being the base component.

In all of the above pharmaceutical compositions, the compounds of the invention are formulated with an acceptable carrier or excipient. The carriers or excipients used must, of course, be acceptable in the sense of being compatible with the other ingredients of the composition and must not be deleterious to the patient. The carrier or excipient can be a solid or a liquid, or both, and is preferably formulated with the compound of the invention as a unit-dose composition, for example, a tablet, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or β-lactose, corn sweeteners, natural and synthetic gums such as acacia, tragacanth or sodium alginate, carboxymethylcellulose, polyethylene glycol, waxes, and the like. Lubricants include sodium oleate, sodium stearate, magnesium stearate, sodium benzoate, sodium acetate, sodium chloride, and the like. Disintegrators include starch, methyl cellulose, agar, bentonite, xanthan gum, and the like.

Pharmaceutically acceptable carriers and excipients encompass all the foregoing additives and the like.

### Examples of Pharmaceutical Formulations

A. TABLETS

| Component | Amount per tablet (mg) |
|---|---|
| active substance | 100 |
| lactose | 140 |
| corn starch | 240 |
| polyvinylpyrrolidone | 15 |
| magnesium stearate | 5 |
| TOTAL | 500 |

The finely ground active substance, lactose, and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B. TABLETS

| Component | Amount per tablet (mg) |
|---|---|
| active substance | 80 |
| lactose | 55 |
| corn starch | 190 |
| polyvinylpyrrolidone | 15 |
| magnesium stearate | 2 |
| microcrystalline cellulose | 35 |
| sodium-carboxymethyl starch | 23 |
| TOTAL | 400 |

The finely ground active substance, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

C. COATED TABLETS

| Component | Amount per tablet (mg) |
|---|---|
| active substance | 5 |
| lactose | 30 |
| corn starch | 41.5 |
| polyvinylpyrrolidone | 3 |
| magnesium stearate | 0.5 |
| TOTAL | 90 |

The active substance, corn starch, lactose, and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

D. CAPSULES

| Component | Amount per capsule (mg) |
|---|---|
| active substance | 50 |
| corn starch | 268.5 |
| magnesium stearate | 1.5 |
| TOTAL | 320 |

The substance and corn starch are mixed and moistened with water. The moist mass is screened and dried. The dry granules are screened and mixed with magnesium stearate. The finished mixture is packed into size 1 hard gelatine capsules.

E. AMPOULE SOLUTION

| Component | Amount per ampoule | |
|---|---|---|
| active substance | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilized and sealed by fusion. The ampoules contain 5 mg, 25 mg, and 50 mg of active substance.

F. SUPPOSITORIES

| Component | Amount per suppository (mg) |
|---|---|
| active substance | 50 |
| solid fat | 1650 |
| TOTAL | 1700 |

The hard fat is melted. At 40° C., the ground active substance is homogeneously dispersed therein. The mixture is cooled to 38° C. and poured into slightly chilled suppository molds.

G. METERING AEROSOL

| Component | Amount |
|---|---|
| active substance | 0.005 |
| sorbitan trioleate | 0.1 |
| Monofluorotrichloromethane | To 100 |
| and difluorodichloromethane (2:3) | |

The suspension is transferred into a conventional aerosol container with a metering valve. Preferably, 50 μL of suspension are delivered per spray. The active substance may also be metered in higher doses if desired (e.g., 0.02% by weight).

H. POWDER FOR INHALATION

| Component | Amount | |
|---|---|---|
| active substance | 1.0 | mg |
| lactose monohydrate | to 25 | mg |

I. POWDER FOR INHALATION

| Component | Amount | |
|---|---|---|
| active substance | 2.0 | mg |
| lactose monohydrate | to 25 | mg |

J. POWDER FOR INHALATION

| Component | Amount | |
|---|---|---|
| active substance | 1.0 | mg |
| lactose monohydrate | to 5 | mg |

K. POWDER FOR INHALATION

| Component | Amount | |
|---|---|---|
| active substance | 2.0 | mg |
| lactose monohydrate | to 5 | mg |

In Examples H, I, J, and K, the powder for inhalation is produced in the usual way by mixing the individual ingredients together.

What is claimed is:

1. A compound of a formula: 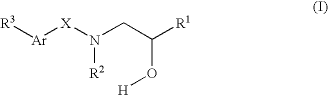
or the pharmaceutically acceptable salts thereof wherein:
R¹ is hydrogen, C₁-C₆ alkyl, C₃-C₁₀ cycloalkyl, aryl or heteroaryl, wherein the aryl or heteroaryl are each optionally substituted with 1-3 substituents; or,
R¹ is C₁-C₃ alkyl substituted with Z-R⁴, wherein Z is O, S, SO₂, NH, NMe or CH₂ and R⁴ is aryl or heteroaryl, wherein the aryl or heteroaryl is optionally substituted with 1-3 substituents;
R² is hydrogen or C₁-C₆ alkyl;
X is a methylene group which is optionally mono- or di-substituted with methyl; or,
X is a carbonyl group;
Ar is a divalent moiety which is either phenylene or a six-membered heteroarylene, which divalent moiety is optionally mono- or disubstituted with moieties selected from the group consisting of C₁-C₆ alkyl optionally substituted by one to 3 halogen atoms, C₃-C₁₀ cycloalkyl and halogen; or
Ar is a fused aromatic ring system optionally containing one or more heteroatoms; and
R³ is H, NR⁵R⁶, OR⁶, SO₂R⁶, or CH₂R⁶, wherein R⁵ is hydrogen or C₁-C₆ alkyl, and R⁶ is aryl or heteroaryl, wherein the aryl or heteroaryl are each optionally substituted with 1-3 substituents.

2. The compound according to claim 1, wherein:
R¹ is hydrogen, C₁-C₆ alkyl, C₃-C₁₀ cycloalkyl, phenyl, or pyridyl;
R² is hydrogen or C₁-C₆ alkyl;
X is a methylene group optionally mono- or disubstituted with methyl; or a carbonyl group;
Ar is as set forth in claim 1; and
R³ is H, NR⁵R⁶, OR⁶, SO₂R⁶, or CH₂R⁶, wherein R⁵ is hydrogen or C₁-C₆ alkyl and R⁶ is an aryl or heteroaryl moiety, wherein the aryl or heteroaryl moiety is optionally substituted with a substituent selected from the group consisting of C₁-C₆ alkyl optionally substituted with 1 to 3 halogen atoms, C₁-C₆ alkoxy optionally substituted with 1 to 3 halogen atoms, C₁-C₆ alkoxycarbonyl, C₁-C₆ alkylaminocarbonyl, C₁-C₆ dialkylaminocarbonyl, hydroxyl, halogen, cyano and nitro.

3. The compound according to claim 1, wherein:
R¹ is phenyl;
R² is hydrogen or C₁-C₆ alkyl;
X is a methylene group;
Ar is a 1,4-phenylene or 1,4-pyridylene; or a quinoline; and
R³ is H, NR⁵R⁶, OR⁶, SO₂R⁶, or CH₂R⁶, wherein R⁵ is hydrogen or methyl and R⁶ is a phenyl, wherein the phenyl is optionally mono- or di-substituted with methyl or chlorine or a combination of methyl and chlorine.

4. The compound according to claim 1 wherein said compound is selected from the group consisting of:
(R)-2-{[4-(2-Chloro-phenylamino)-benzyl]-methyl-amino}-1-phenyl-ethanol;
2-{[6-(2-Chloro-phenylamino)-pyridin-3-ylmethyl]-methyl-amino}-1-phenyl-ethanol;
2-[Methyl-(4-o-tolylamino-benzyl)-amino]-1-phenyl-ethanol;
2-[Methyl-(6-o-tolylamino-pyridin-3-ylmethyl)-amino]-1-phenyl-ethanol;
2-{[4-(2-Chloro-phenylamino)-benzyl]-methyl-amino}-1-phenyl-ethanol;
(R)-2-[(4-Benzenesulfonyl-benzyl)-methyl-amino]-1-phenyl-ethanol;
(R)-2-[Methyl-(4-phenylamino-benzyl)-amino]-1-phenyl-ethanol;
2-[Methyl-(4-phenylamino-benzyl)-amino]-1-phenyl-ethanol;
2-{[6-(5-Chloro-2-methyl-phenylamino)-pyridin-3-ylmethyl]-methyl-amino}-1-phenyl-ethanol;
(R)-2-[Methyl-(4-phenoxy-benzyl)-amino]-1-phenyl-ethanol;
2-{[6-(2-Chloro-5-methyl-phenylamino)-pyridin-3-ylmethyl]-methyl-amino}-1-phenyl-ethanol;
2-{[6-(2,5-Dimethyl-phenylamino)-pyridin-3-ylmethyl]-methyl-amino}-1-phenyl-ethanol;
(R)-2-(methyl-quinolin-3-ylmethyl-amino)-1-phenyl-ethanol;
2-{[4-(2,5-Dimethyl-phenylamino)-benzyl]-methyl-amino}-1-phenyl-ethanol;
(S)-2-[Methyl-(6-phenoxy-pyridin-3-ylmethyl)-amino]-1-phenyl-ethanol;
2-{[4-(2-chloro-5-methyl-phenylamino)-benzyl]-methyl-amino}-1-phenyl-ethanol;
(S)-1-Phenyl-2-[(quinolin-3-ylmethyl)-amino]-ethanol;
and the salt thereof.

5. The compound according to claim 1 wherein said compound is selected from the group consisting of:
(R)-2-{[4-(2-Chloro-phenylamino)-benzyl]-methyl-amino}-1-phenyl-ethanol;
2-{[6-(2-Chloro-phenylamino)-pyridin-3-ylmethyl]-methyl-amino}-1-phenyl-ethanol;
2-[Methyl-(4-o-tolylamino-benzyl)-amino]-1-phenyl-ethanol;
2-[Methyl-(6-o-tolylamino-pyridin-3-ylmethyl)-amino]-1-phenyl-ethanol;
2-{[4-(2-Chloro-phenylamino)-benzyl]-methyl-amino}-1-phenyl-ethanol;
(R)-2-[(4-Benzenesulfonyl-benzyl)-methyl-amino]-1-phenyl-ethanol;
(R)-2-[Methyl-(4-phenylamino-benzyl)-amino]-1-phenyl-ethanol;
2-[Methyl-(4-phenylamino-benzyl)-amino]-1-phenyl-ethanol;
2-{[6-(5-Chloro-2-methyl-phenylamino)-pyridin-3-ylmethyl]-methyl-amino}-1-phenyl-ethanol;
(R)-2-[Methyl-(4-phenoxy-benzyl)-amino]-1-phenyl-ethanol;
2-{[6-(2-Chloro-5-methyl-phenylamino)-pyridin-3-ylmethyl]-methyl-amino}-1-phenyl-ethanol;
2-{[6-(2,5-Dimethyl-phenylamino)-pyridin-3-ylmethyl]-methyl-amino}-1-phenyl-ethanol;
and the salt thereof.

6. A process for preparing a compound of a formula 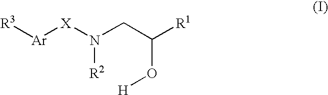
according to claim 1 comprising:
reacting a starting material of a formula 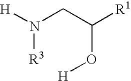
with an aldehyde of formula Y—Ar—CHO (Y is Cl, F, or Br) or a ketone, in a solvent, in the presence of a reducing agent to provide an alkylated amine of a formula 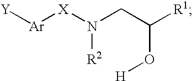
or, reacting the starting material with a halide of formula Y—Ar—CH₂-Hal wherein Hal is Cl, Br or I, in a solvent, in the presence of a base to provide the alkylated amine; and
reacting the alkylated amine with an amine of formula R⁵R⁶NH in the presence of a base with or without palladium catalyst to provide a compound of formula (I) wherein R³ is —NR⁵R⁶; or
reacting the alkylated amine with a phenol of formula R⁶OH, in a solvent, in the presence of a base and copper iodide to provide a compound of formula (I) wherein R³ is —OR⁶; or
reacting the alkylated amine with a sulfonyl chloride of formula R⁶SO₂Cl, in a solvent, in the presence of a base to provide a compound of formula (I) wherein R³ is —SO₂R⁶.

7. A method for the treatment of a CB2 receptor-mediated disease or condition in an animal subject comprising administering to said animal subject in need of such treatment a therapeutically effective dose of the compound according to claim 1 or a pharmaceutically acceptable salt thereof.

8. The method according to claim 7 wherein said CB2 receptor-mediated disease or condition is selected from the group consisting of an inflammatory disease and an autoimmune disease.

9. The method according to claim 7 wherein said CB2 receptor-mediated disease or condition is pain.

10. The method according to claim 7 wherein said CB2 receptor-mediated disease or condition is a lung disease, a rheumatic disease, an autoimmune disease, a musculoskeletal disease, an allergic disease, an allergic reaction, a vascular disease, a dermatological disease, a renal disease, a hepatic disease, a gastrointestinal disease, neurodegeneration eye disease, diseases of the ear, nose, and throat, neurological disease blood disease, tumors, endocrine diseases, organ and tissue transplantations and graft-versus-host diseases, severe states of shock, acute pain, visceral pain, spasm of the gastrointestinal tract or uterus, colics, neuropathic pain, inflammatory and nociceptive pain, cancer pain, headache, restenosis, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis, and pyrexia.

11. A pharmaceutical composition comprising the compound according to claim 1 or a pharmaceutically acceptable salt thereof and a pharmaceutically acceptable carrier.

12. Use of the compound according to claim 1 in the manufacture of a medicament for treatment of a CB2 receptor-mediated disease or condition.

13. The use according to claim 12 wherein said CB2 receptor-mediated disease or condition is selected from the group consisting of an inflammatory disease and an autoimmune disease.

14. The use according to claim 12 wherein said CB2 receptor-mediated disease or condition is pain.

15. The use according to claim 12 wherein said CB2 receptor-mediated disease or condition is a lung disease, a rheumatic disease, an autoimmune disease, a musculoskeletal disease, an allergic disease, an allergic reaction, a vascular disease, a dermatological disease, a renal disease, a hepatic disease, a gastrointestinal disease, neurodegeneration eye disease, diseases of the ear, nose, and throat, neurological disease blood disease, tumors, endocrine diseases, organ and tissue transplantations and graft-versus-host diseases, severe states of shock, acute pain, visceral pain, spasm of the gastrointestinal tract or uterus, colics, neuropathic pain, inflammatory and nociceptive pain, cancer pain, headache, restenosis, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis, and pyrexia.
